(19) United States
(12) Patent Application Publication
(10) Pub. No.: US 2007/0255163 A1
(43) Pub. Date: Nov. 1, 2007

(54) Journey event monitor and recording apparatus

(76) Inventor: Frank Joseph Prineppi, Freeport (BS)

Correspondence Address: FRANK PRINEPPI;PMB 341, 1323 SE 17th ST., FT. LAUDERDALE, FL, 33316, US

(21) Appl. No.: 11/401,848

(22) Filed: Apr. 12, 2006

(51) Int. Cl. A61B 5/00

(52) U.S. Cl. 600/549

(57) ABSTRACT

Apparatus for monitoring events such as the measurement of temperature, pressure, humidity or physical shock caused by acceleration or deceleration is disclosed. The apparatus includes one or more sensors for measuring the events and circuitry for recording these events and displaying the same along with a time of day and a date when the event occurred. An additional alarm e.g. a visual or audible alarm is provided to warn of an impending problem such as a thaw of frozen items. The alarm is operated when a thaw condition is possible. A display in the form of a series of bar graphs is disclosed for displaying a series of event measurements.

### BACKGROUND OF THE INVENTION

This invention relates to apparatus for monitoring and recording physical parameters and events, especially but not exclusively, during the transportation of products, such as by containerised ship transport.

It is well known to provide apparatus and methods for monitoring and recording events during a journey, a good example being the “black box” flight recorder fitted to most aircraft, which constantly monitors in-flight events both Within and without the aircraft, generally from inputs such as speed, altitude, temperature, pressure and so on, as well as engine performance, fuel capacity, structural integrity and so on. However, such journey logging systems are extremely complicated and expensive to both make and install such that they are inappropriate for adaptation to general use for monitoring and recording events during e.g. a sea voyage of a single container carried by a container ship. On the other hand, for some products such as food it is often desirable to know what temperature extremes have been experienced at sea or during transportation over land, and for delicate products, such as electro-optical apparatus, it is often convenient to know what G-forces the container has been subjected to during such a journey, each for the purposes of showing how badly or well the container was treated, or for the purposes of obtaining evidence for claiming on insurance against damage.

### SUMMARY OF THE INVENTION

The present invention is derived from the realization that there is a need for a portable, lightweight journey monitoring apparatus to be provided which is relatively inexpensive, preferably highly visible, so as to alert those involved in the handling of e.g. a shipping container that journey events are being monitored and recorded, and which can be easily interrogated during and/or after completion of the journey.

According to the invention there is provided a portable electric event monitoring recording and display (“MRD”) apparatus comprising: a housing, monitor means within the housing to continuously or sequentially monitor events selected from a group consisting of the measurement of one or more of temperature, pressure, humidity and physical shock, recording means to record the or each event, interrogator means to interrogate the record and visual display means for displaying one or more such event records over time.

Conveniently, the MRD apparatus includes a warning device, such as audible warning means, upon the happening of an event requiring attention, such as an unacceptably high rise in temperature indicative of potential melt down of the product in transit, whereafter remedial action may be taken to restore refrigeration.

Conveniently, the MRD apparatus includes one or more sensors, which may preferably protrude from the housing, to sense events such as temperature and humidity, and there may also be one or more remote sensors communicable directly or indirectly to the recording means of the apparatus to thereby sense events remote from the housing, a particularly useful feature when covering large areas such as the inside of containers.

Preferably, the housing is disposed within an airtight container during use.

Preferably, the housing is completely surrounded by said container with no physical connections to the housing being accessible from outside the container.

### BRIEF DESCRIPTION OF THE DRAWINGS

One illustrative embodiment of the invention will now be described with reference to the drawings, wherein:

FIG. 1 shows MRD apparatus according to the invention,

FIG. 2 shows a section of the apparatus shown in FIG. 1,

FIG. 3 shows a partial view of a further embodiment of the apparatus according to the invention,

FIG. 4 shows a further section of the apparatus shown in FIG. 3,

FIG. 5 shows a section of a modified version of the apparatus shown in FIG. 3, and

FIG. 6 shows a schematic circuit diagram for the embodiments of FIG. 1 or 3.

### DETAILED DESCRIPTION OF THE DRAWINGS

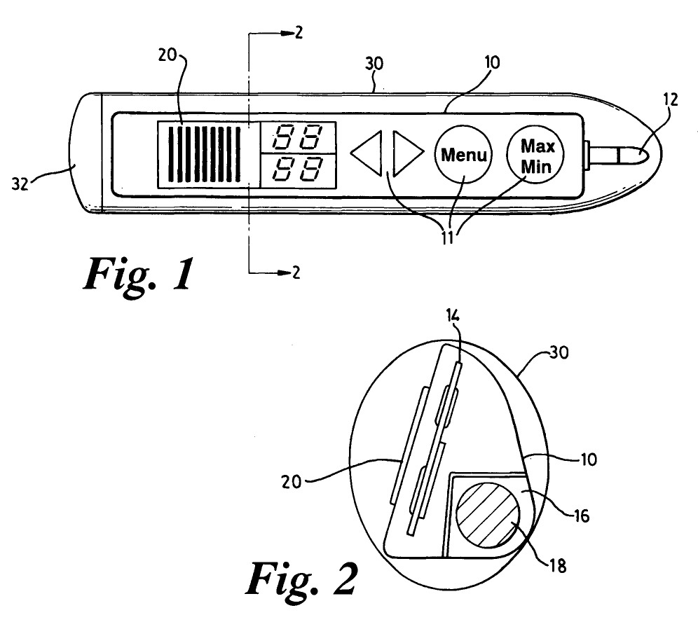

FIG. 1 shows an event monitoring, recording and display (MRD) apparatus comprising a housing 10 which houses an electronic monitor for continuously and sequentially monitoring events such as changes in ambient conditions, e.g. temperature, pressure, humidity, or physical shock. The housing includes also means for recording a series of such events, a means to interrogate the record and a display means 20 for displaying one or more such event records.

The housing 10 is contained within a transparent acrylic tube-shaped protective container 30. The container 30 has a resilient removable bung 32 at one end to form an airtight seal and thereby protect the MRD apparatus from the ingress of water or other contaminants during monitoring. The container 30 encloses also a temperature probe 12 which projects from the housing 10, and thereby completely surrounds the apparatus so that it is hermetically sealed within the container. The bung 32 can be removed to inspect the apparatus.

FIG. 2 shows a section on the line 2-2 in FIG. 1. In addition to the components shown in FIG. 1, FIG. 2 shows a printed circuit board (pcb) 14 on which is mounted the electronic components of the apparatus. Additionally, a battery compartment 16 is shown which houses batteries 18.

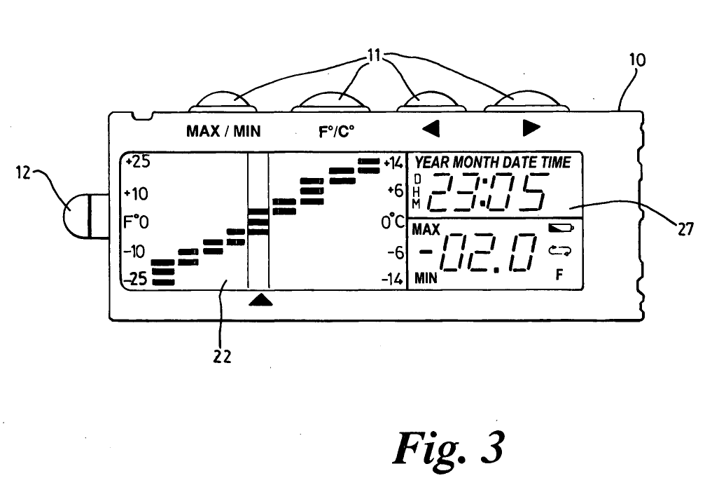

FIG. 3 shows a partial, enlarged view of another embodiment of the apparatus, parts in common with the embodiment shown in FIG. 1 having like reference numerals. In this embodiment the buttons 11 are on top of the housing 10. With the aid of buttons 11 (operable in a manner similar to those shown in FIG. 1) it is possible to display recorded data. In particular, it is possible to show a series of recorded events (e.g. temperature recordings) in the form of a scrolling bar graph using bar graph elements 22. Also, a maximum and minimum temperature can be displayed together with the time and date that event recording was made. Amongst other functions this embodiment allows a user to:

Display in numeric format maximum or minimum temperatures, based on the maximum or minimum temperature over a selected recording period, and the time and/or date at which that maximum or minimum occurred;

Scroll back and forth through temperature data displayed on display bar graph elements 22, as well as on numeric display elements 27;

Select a temperature threshold in order to display the difference between a temperature threshold and the measured temperature (i.e. the relative temperature difference);

Select a recording mode e.g. the last 100 events, or recording of a fixed number of events e.g. the first 100 events;

Select the recording period e.g. every minute, hour, day or week; and

Reset the apparatus.

In this embodiment no protective container 30 is necessary because the housing 10 is waterproof. However, for hostile environments a protective container 30 may still be used.

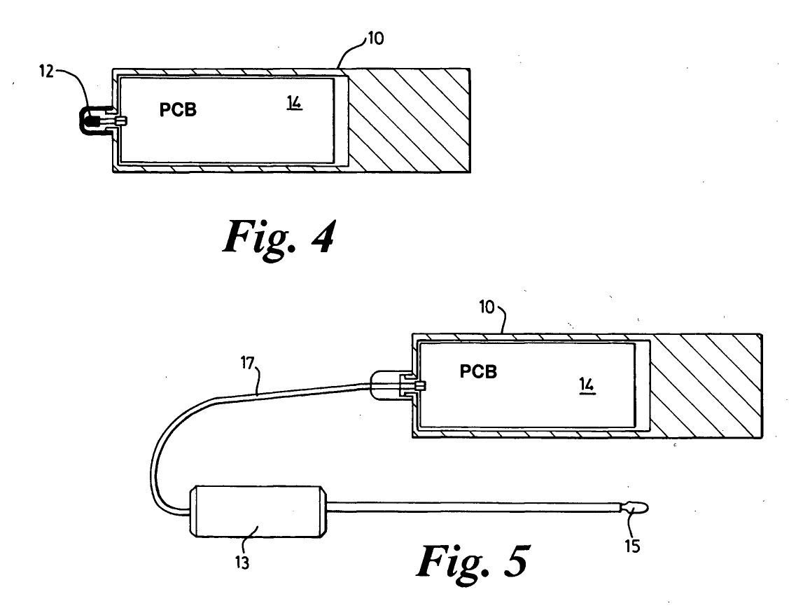

FIG. 4 shows an exposed sectional view through the apparatus shown in FIG. 3. The positioning of the printed circuit board 14 relative to the probe 12 can be seen. The temperature probe 12 is shown having a thermistor connected to the pcb 14.

FIG. 5 shows a modified version of the apparatus shown in FIG. 4. A remote temperature probe 13 is connected to the pcb 14 via a connecting lead 17. A thermistor 15 extends from the probe 13. In this instance it would be necessary for the probe 13 to be located outside the protective container 30, although the container 30 will provide a seal around lead 17 if it is used. The thermistors shown in FIGS. 4 and 5 could, of course, be replaced by other types of temperature sensitive devices.

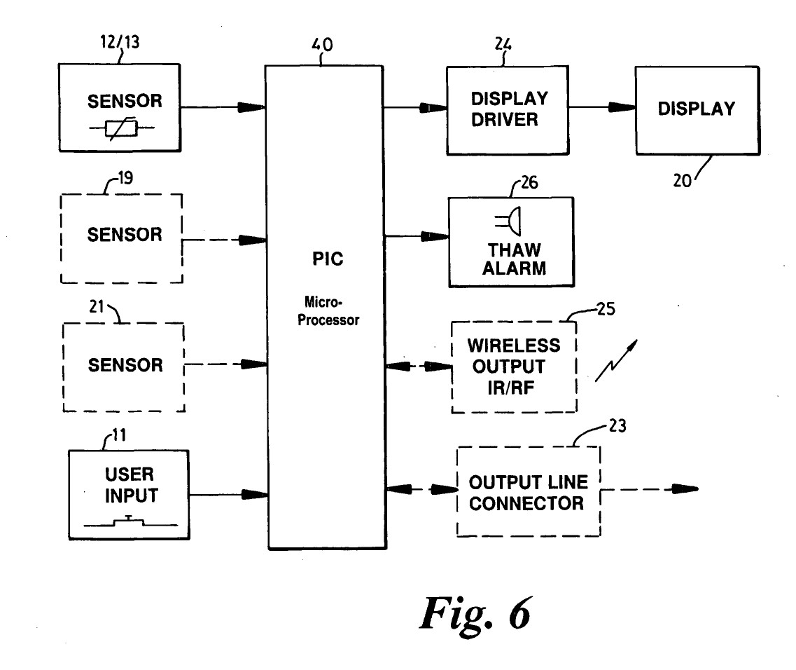

FIG. 6 shows a schematic diagram of the components used within the apparatus shown in FIGS. 1 or 3 which in use will be mounted within housing 10 on pcb 14. The circuit includes input devices in the form of at least one sensor 12/13 and user input means in the form of switches 11 to control the apparatus and to control the display 20. The circuit includes a microprocessor in the form of a programmable interface circuit (PIC) 40 which is programmable to control the operation of the device. The circuit includes also a display driver 24 for converting the output of the PIC 40 into an input for the display 20, although a PIC with a ‘built-in’ display driver could be used instead. Further, an audible alarm 26 is included for giving immediate warning of e.g. a dangerously high temperature being reached.

In operation the apparatus of FIG. 1 or 3 accepts inputs from the temperature sensor 12/13 and optionally further inputs of ambient conditions such as humidity, atmospheric pressure, or other inputs, for example acceleration or deceleration caused by shock loads exerted on the apparatus, from further optional sensors 19 and 21 or other sensors (not shown). The PIC 40 has an internal timer and memory register which, in this instance, records these events in a scrolling manner keeping 100 entries before new data displaces the oldest data. In this way up to 100 of the latest events can be recorded. In this instance, the PIC is programmed to record at regular intervals a maximum or minimum event value that occurred since the last recording and the time and/or date when the recording was made. In this specification the use of the word time includes time of day and/or date. User input switches 11 allow a user to view recorded data in the PIC on the display 20 via the display driver 24. This data can be in the format of a scrolling bar graph type display 22 and the minimum or maximum values can be displayed in conventional numeric form also, at numeric display elements 27. The bar graph is an expanded scale floating type with an auto-centring. The centre element of the display 22 is also displayed in the numeric form. Thus it is possible to display the maximum or minimum values for each period of recording and so to determine the ambient history of the apparatus with the minimum of data storage.

In addition to the visual display an audible alarm 26 can be activated when a predetermined threshold is crossed. In this instance the audible alarm is a thaw alarm to warn of impending thaw of frozen products adjacent the apparatus, or adjacent sensor 13 if this sensor is located remotely from the housing 10. The time of activation of the thaw alarm can be recorded also. In this embodiment the alarm is activated only if the measured temperature has risen above a threshold temperature and that temperature is sustained for a predetermined period (e.g. 2 to 4 hours). If the measured temperature drops below the threshold before the end of the time period then the alarm is not activated and the time period will start again should the temperature rise above the threshold once more.

The PIC 40 can be programmed to allow display of the measurement units to be selected from a choice of metric or imperial. Additionally, Celsius or Fahrenheit temperatures can be displayed on the display. As well as or instead of the display the apparatus may have a physical connection 23 or wireless connection 25 to a data acquisition device such as a PC, PDA or similar device. Such a wireless connection 25 could be via an infrared USB type link, an RF Bluetooth type link or a temporary connecting wire plugged into a socket in the housing 10. The data acquired from the PIC 40 can be stored for use for quality control purposes or the like. The connection can be a two-way link providing re-programming or re-setting information to the PIC 40.

In order to provide a tamper proof apparatus it is envisaged that the programming of the PIC 40 will be encrypted, to prevent manipulation of the data recorded in the register of the PIC. This prevents unauthorized alteration of the recorded data. The PIC can also include a re-set function, which allows the data to be cleared so that the apparatus can be re-used.

Although the invention described above is particularly useful for monitoring, recording and displaying journey event information it will be apparent that such MRD apparatus may also be used in normally stationary surroundings such as the inside of a freezer where, typically, the “journey event” may simply be continuous monitoring, recording and displaying information about the temperature over a continuous period, such as a 100 day period. This is a particularly useful feature in circumstances where there may be disruptions to electric power supply due to weather conditions including hurricanes where it may not be possible or convenient to otherwise ascertain the thermal history of frozen goods within the freezer to see if the food is still safe for consumption.

The MRD apparatus may be used in other applications such as laboratory testing, industrial plant commissioning, workshop projects, agriculture, horticulture, aquiculture, animal husbandry, and the brewing industry.

Another particularly useful feature of the invention is a continuously operable display which advertises the fact that journey events are being monitored and recorded, thereby making it less likely that goods in transit will be mistreated either through neglect or incorrect handling.

The apparatus may be supplemented with a visible warning means external to the goods in transit, such as on the outside of a shipping container, in order to alert the carrier of the goods that recording of treatment in transit is taking place. The visible warning means may take the form of a brightly colored label adhesively affixed to packaged or containerised goods having pre-printed warnings thereon.

Two embodiments of the invention only have been described and illustrated. However, it will be apparent to the skilled addressee that further modifications, variants, equivalents and adaptations are possible without departing from the spirit or scope of the invention. For example, the circuit shown in FIG. 6 may be replaced by an application specific integrated circuit (ASIC) or a programmable gate array (PGA) such as a mask or field PGA, or some other equivalent circuit.

1. A portable electric event monitoring recording and display (“MRD”) apparatus comprising: a housing, monitor means within the housing to continuously or sequentially monitor events selected from a group consisting of the measurement of one or more of temperature, pressure, humidity and physical shock, recording means to record the or each event, interrogator means to interrogate the record and visual display means for displaying one or more such event records over time.

2. MRD apparatus as claimed in claim 1, including a warning device, such as an audible warning device, operable upon the happening of an event requiring attention, such as the measurement of an unacceptably high temperature indicative of potential melt down of the product in transit.

3. MRD apparatus as claimed in claim 1 wherein the apparatus includes one or more sensors, which measure the events.

4. MRD apparatus as claimed in claim 3 wherein the or each sensor protrudes from the housing.

5. MRD apparatus as claimed in claim 3 wherein the or each sensor is remote from the housing and is communicable directly or indirectly to the recorder to measure events remote from the housing.

6. MRD apparatus as claimed in claim 1 wherein the housing is disposed within an airtight container during use.

7. MRD apparatus as claimed in claim 6 wherein the housing is completely surrounded by said container with no physical connections to the housing being accessible from outside the container.

8. MRD apparatus as claimed in claim 1 wherein the events are recorded during a pre-selected time period and the maximum or minimum event measurement for that time period is recorded.

9. MRD apparatus as claimed in claim 8 wherein the further recorded maximum or minimum event measurement is displayable on the visual display.

10. MRD apparatus as claimed in claim 8 wherein the apparatus further includes a timer, and the recording means records the time of day and/or the date at which the events are recorded.

11. MRD apparatus as claimed in claim 2 wherein the apparatus further includes a timer, and the recording means records the time of day and/or the date at which the event requiring attention is recorded.

12. MRD apparatus as claimed in claim 11 wherein the event requiring attention is a rise in measured temperature from a temperature threshold below approximately −4 to −2 degrees Celsius to a higher measured temperature above that threshold and the maintaining of that higher temperature above that threshold for a period of time.

13. MRD apparatus as claimed in claim 12 wherein a reduction in the measured temperature to a temperature below said threshold before the expiry of said period of time prevents the operation of the warning device.

14. MRD apparatus as claimed in claim 1 wherein the visual display means includes a bar graph display comprising a plurality of adjacent bar graph elements and the apparatus further includes a display circuit operable for displaying on each element a sequence of the measurement values of the events in a graphical form.

15. MRD apparatus as claimed in claim 14 wherein the visual display means further includes a numeric display and the display circuit is further operable to display said measurement value of one of the events in numeric form and to display the time of day and/or date at which that numerically displayed measurement value was recorded.
